(19) United States
(12) Patent Application Publication
(10) Pub. No.: US 2024/0041459 A1
(43) Pub. Date: Feb. 8, 2024

(54) ANTI-MIGRATION FEATURES OF DEVICES, SYSTEMS, AND METHODS FOR CREATING AN ANASTOMOSIS

(71) Applicant: BOSTON SCIENTIFIC SCIMED, INC., MAPLE GROVE, MN (US)

(72) Inventors: Fionn Stapleton, Galway (IE); John Thomas Favreau, Shrewsbury, MA (US); Ryan Desmond Lynch, Roscommon (IE); Sandra Burke, Galway (IE); Michael Walsh, Corofin (IE); Francis Power, Galway (IE); Sean P. Fleury, Princeton, MA (US)

(73) Assignee: BOSTON SCIENTIFIC SCIMED, INC., MAPLE GROVE, MN (US)

(21) Appl. No.: 18/228,767

(22) Filed: Aug. 1, 2023

Related U.S. Application Data
(60) Provisional application No. 63/394,477, filed on Aug. 2, 2022.

(51) Int. Cl. A61B 17/11

(52) U.S. Cl. CPC A61B 17/11; A61B 2017/1139

(57) ABSTRACT

An implantable medical device with at least a portion of a wall thereof being coated to prevent fluid flow therethrough, and having one or more uncoated anti-migration features promoting tissue ingrowth therearound. The anti-migration features may be partially-closed or fully closed shapes forming a retention portion transverse to the direction along which migration forces typically impact the implantable medical device at the anatomical site at which the implantable medical device is to be deployed.

### CROSS-REFERENCE TO RELATED APPLICATIONS

This application claims the benefit of priority under 35 U.S.C. § 119 to U.S. Provisional Application No. 63/394,477, filed Aug. 2, 2022, the entire disclosure of which is hereby incorporated by reference herein for all purposes.

### FIELD

The present disclosure relates generally to the field of implantable medical devices. In particular, the present disclosure relates to medical devices, systems, and methods extending across anatomical structures, such as for establishing a connection and/or fluid communication between the anatomical structures. More particularly, the present disclosure relates to anti-migration features of devices, systems, and methods for establishing a connection and/or fluid communication between anatomical structures.

### BACKGROUND

Various devices such as stents are known for extending across anatomical structures for various purposes. For instance, various stents are known for establishing connections between anatomical structures. Some such connections are made simply to hold tissue in apposition, whereas some such connections also establish fluid communication between anatomical structures such as organs, cavities, lumens, passages, etc. In some instances, it is desirable to create a semi-permanent or permanent anastomosis allowing fluid flow or drainage from one anatomical structure to another anatomical structure. For example, in various gastrointestinal (GI) procedures (e.g., gastric bypass surgery), lumen apposing stents may be used to form an anastomosis in the GI system, such as a gastrojejunostomy between the stomach and the jejunum. The gastrojejunostomy facilitates flow of food particulate, liquid, chyme, etc., from the stomach to the lower GI tract, bypassing the pylorus and the duodenum (e.g., approximately the first 1.5 m of the small intestine, where most food, fats, and nutrients are digested). Such procedure is considered to be less invasive than prior Roux-en-Y surgical bypass procedures, and may be reversible. As may be appreciated, it is desirable for the anastomosis device to remain securely in place until removal is desired or medically indicated. In general, in various procedures or uses of a stent extending across anatomical structures, such as to create an anastomosis, it may desirable for the stent to remain in place for a prolonged period of time (e.g., days, weeks, months, even upwards of six to twelve months). Accordingly, there is an ongoing need for associated devices, systems, and methods with more robust anti-migration features.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an implantable medical device includes an elongate body having a proximal end and a distal end and defining a lumen extending therethrough; a proximal retention member along the proximal end of the elongate body; a distal retention member along the distal end of the elongate body; a saddle region defined between the proximal retention member and the distal retention member; and at least one anti-migration feature extending outwardly from an outer surface of the elongate body. In some aspects, the at least the saddle region has a wall with a coating formed of a material preventing flow of fluid therethrough; and the at least one anti-migration feature is uncoated to promote tissue ingrowth therearound.

In some embodiments, the at least one anti-migration feature is in the form of a semi-closed or closed shape with respect to the elongate body. In some embodiments, the at least one anti-migration feature includes at least one retention portion extending transverse to an axis along which migratory forces impact the implantable medical device.

In some embodiments, the lumen of the elongate body extends through the saddle region and the retention portion extends transverse to the longitudinal axis of the saddle region. In some embodiments, the at least one anti-migration feature extends from and transverse to at least one of the proximal retention member or the distal retention member in a direction toward the saddle region.

In some embodiments, the proximal retention member and the distal retention member extend radially outwardly from the saddle region; and the at least one anti-migration feature extends from and transverse to at least one of the proximal retention member or the distal retention member in a direction toward the saddle region.

In some embodiments, the saddle region is configured to extend between a proximal tissue wall and a distal tissue wall, the proximal retention member is configured to anchor the implantable medical device with respect to the proximal tissue wall, the distal retention member is configured to anchor the implantable medical device with respect to the distal tissue wall, and the at least one anti-migration feature is configured to embed into one of the proximal tissue wall or the distal tissue wall.

In some embodiments, the at least one anti-migration feature includes at least one anti-migration feature extending from the proximal retention member toward the saddle region and at least one anti-migration feature extending from the distal retention member toward the saddle region.

In some embodiments, the implantable medical device is shiftable between an elongated delivery configuration and a foreshortened deployed configuration; the proximal retention member and the distal retention member are defined upon the implantable medical device shifting to the deployed configuration and a portion of the elongate body extending radially outward; in the foreshortened configuration, the length of the saddle region and the configuration of the proximal retention member and the distal retention member are selected to draw together tissues across which the elongate body is extended, with the at least one anti-migration feature anchoring into the tissues.

In some embodiments, at least one of the proximal retention member or the distal retention member is formed from woven filaments, and the at least one anti-migration feature is formed from an extension of one of the woven filaments.

In some embodiments, the at least one anti-migration feature is separately formed from and coupled to at least one of the proximal retention member or the distal retention member.

In accordance with various principles of the present disclosure, an implantable medical device includes an elongate body having a proximal end and a distal end and defining a lumen extending therethrough; and at least one anti-migration feature extending outwardly from an outer surface of the elongate body. In some aspects, the elongate body is formed from a plurality of filaments forming a wall of the elongate body with interstices therethrough; at least a portion of the wall of the elongate body is coated to prevent flow of fluid therethrough and to resist tissue ingrowth therein; and the at least one anti-migration feature is uncoated to promote tissue ingrowth therearound.

In some embodiments, the at least one anti-migration feature is in the form of a semi-closed or closed shape with respect to the elongate body. In some embodiments, the at least one anti-migration feature includes at least one retention portion extending transverse to an axis along which migratory forces impact the implantable medical device.

In some embodiments, the implantable medical device further includes a proximal retention member extending radially outwardly along the proximal end of the elongate body, a distal retention member extending radially outwardly along the distal end of the elongate body, and a saddle region defined between the proximal retention member and the distal retention member; the at least one anti-migration feature extends from at least one of the retention members towards the saddle region.

In accordance with various principles of the present disclosure, a method for creating an anastomosis includes extending an implantable medical device across a proximal tissue wall and a distal tissue wall; allowing a proximal end of the implantable medical device to expand radially outwardly with respect to a saddle region extending through the tissue walls to form a proximal retention member anchored against a proximal side of the proximal tissue wall; and allowing a distal end of the implantable medical device to expand radially outwardly with respect to the saddle region to form a distal retention member anchored against distal side of the distal tissue wall. In some aspects, the saddle region has a tubular wall defining a lumen therethrough, the wall being coated with a material preventing passage of fluid therethrough; and the method further includes positioning the implantable medical device so that at least one uncoated anti-migration feature extends towards at least one of the proximal tissue wall or the distal tissue wall and tissue ingrowth with respect to the at least one anti-migration feature is promoted.

In some embodiments, the method further includes causing the at least one anti-migration feature to embed into the at least one of the proximal tissue wall or the distal tissue wall.

In some embodiments, the method further includes deploying the implantable medical device such that the proximal retention member and the distal retention member draw the proximal tissue wall and the distal tissue wall together in apposition to facilitate formation of an anastomosis therebetween.

In some embodiments, the method further includes positioning the implantable medical device so that at least one uncoated anti-migration feature extends from at least one of the proximal retention member or the distal retention member and towards a respective proximal or distal tissue wall to promote tissue ingrowth with respect to the at least one uncoated anti-migration feature.

In some embodiments, the method further includes positioning the implantable medical device so that at least one proximal uncoated anti-migration feature extends transverse to and from the proximal retention member to the proximal tissue wall to promote tissue ingrowth with respect to the at least one proximal uncoated anti-migration feature, and so that at least one distal uncoated anti-migration feature extends transverse to and from the distal retention member and towards the distal tissue wall to promote tissue ingrowth with respect to the at least one distal uncoated anti-migration feature.

In accordance with various principles of the present disclosure, a method for forming an implantable medical device having a lumen defined therethrough with at least one anti-migration feature is disclosed. In some embodiments, the method includes coating the wall of the implantable medical device, defining the lumen through the implantable medical device with a material to prevent passage of fluid through the wall; and extending an uncoated filament outwardly from an outer surface of the implantable medical device and in an at least partially-closed shape to form an anti-migration feature promoting tissue ingrowth therearound.

In some embodiments, the method further includes extending at least a portion of the uncoated filament transverse to the longitudinal axis of the implantable medical device to form an uncoated retention portion of the anti-migration feature.

In some embodiments, the method further includes fully coating the walls of the implantable medical device without coating the anti-migration feature.

In some embodiments, the method further includes extending the uncoated filament from a portion of the wall of the implantable medical device expandable into a retention member with a diameter greater than an adjoining saddle region of the implantable medical device. In some embodiments, the method further includes extending the uncoated filament toward the saddle region.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. illustrates a perspective view of an embodiment of an implantable medical device formed in accordance with various aspects of the present disclosure and positioned in a schematic representation of a gastrointestinal environment.

FIG. 2 illustrates an elevational view of an implantable medical device formed in accordance with various principles of the present disclosure.

FIG. 3A illustrates an elevational view of an embodiment of an implantable medical device formed in accordance with various aspects of the present disclosure and positioned across a schematic representation of apposed tissue walls.

FIG. 3B illustrates an elevational view of an embodiment of an implantable medical device as in FIG. 3A, but with the tissue walls forming an anastomosis.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “channel” or “bore” or “lumen” or “passage” is not limited to a circular cross-section. As used herein, a “free end” of an element is a terminal end at which such element does not extend beyond. Finally, reference to “at” a location or site is intended to include tissue at and/or about the vicinity of (e.g., along, adjacent, etc.) such location or site.

In accordance with various principles of the present disclosure, implantable medical devices are formed to extend across adjacent or apposed anatomical structures. It will be appreciated that such implantable medical devices may be referenced herein as scaffolds, grafts, stents, etc., without intent to limit. In accordance with various further principles of the present disclosure, such implantable medical devices are formed to hold anatomical structures in apposition. Even more particularly, such stents may be formed to establish a flow or access passage between the apposed anatomical structures. The anatomical structures may be lumens, channels, vessels, passages, cavities, organs, cysts, pseudocysts, etc., the present disclosure not necessarily being limited to use between particular anatomical structures. For the sake of convenience, and without intent to limit, reference may be made to holding tissue walls in apposition, it being appreciated that such is only one example of anatomical structures and association therewith for which principles of the present disclosure are applicable.

An example of a use of an implantable medical device formed in accordance with various principles of the present disclosure is to form an anastomosis between a patient's stomach and a section of the patient's small intestines, such as the jejunum (also known as a gastrojejunostomy). For the sake of convenience, and without intent to limit, the foregoing description of devices, systems, and methods is made with reference to formation of a gastrojejunostomy, but the present disclosure should be understood as not being limited to only such use or application. Moreover, it will be appreciated that although the present disclosure refers to application to the gastrointestinal system, principles of the present disclosure may be applied to other systems or structures of a patient's body as may be appreciated by those of ordinary skill in the art.

An implantable medical device formed in accordance with various principles of the present disclosure includes an elongate body shiftable from a delivery configuration to a deployed configuration. In the delivery configuration, the elongate body is generally compact and/or constricted to be capable of transcatheter delivery through a patient's body without requiring an open surgical procedure. For instance, the implantable medical device may be delivered via a natural orifice transluminal endoscopic surgery (NOTES) procedure, considered to be simpler and less invasive than open surgical procedures (such as Roux-en-Y procedures). Accordingly, in the delivery configuration, the implantable medical device may be compressed and/or elongated or otherwise configured to be able to fit within a generally tubular delivery device (e.g., endoscope, catheter, shaft, etc.) capable of transluminal delivery through the patient's body. Once the implantable medical device is delivered to the desired anatomical site (which may be alternately referenced herein as a treatment site, deployment site, delivery site, etc., without intent to limit), the implantable medical device may be allowed to shift into a deployed configuration. In the deployed configuration, the implantable medical device may be in a generally expanded configuration. In the deployed configuration, the implantable medical device may define a saddle region with a first end and a second end and one or more retention members (which may alternately be referenced herein as a flange) at or along each end thereof. It will be appreciated that terms such as at or on or adjacent or along an end may be used interchangeably herein without intent to limit unless otherwise stated, and are intended to indicate a general relative spatial relation rather than a precisely limited location. The retention members are sized, shaped, configured, and/or dimensioned to retain the implantable medical device with respect to the deployment site. More particularly, the size, shape, configuration, and/or dimensions of the retention members may be selected to seat against a body wall extending radially outwardly from the body passage through which the saddle region of the implantable medical device is positioned. As such, the retention members are transverse to, and typically extend substantially perpendicular to, the saddle region of the implantable medical device. Typically, the retention members are wider (in a radial direction transverse to the longitudinal axis of the body passage) than the saddle region. It will be appreciated that reference to a body passage includes naturally-existing passages (e.g., the pylorus) as well as medically-created passages (e.g., a passage created with the use of a medical instrument, such as between a stomach and jejunum) or otherwise.

In some aspects of the present disclosure, the saddle region defines a lumen therethrough to allow passage of materials (e.g., fluid) from one anatomical structure, through the lumen of the saddle region, and to another anatomical structure. The retention members of the implantable medical device retain the implantable medical device in place with respect to the two anatomical structures. Additionally or alternatively, the retention members hold in apposition the tissue of the anatomical structures between/across which the implantable medical device is positioned. In accordance with various principles of the present disclosure, the implantable medical device, including the saddle region and retention members, is partially or fully coated with a material which prevents passage of material through the walls thereof, such as through the wall of the saddle region. Such coating typically inhibits tissue ingrowth into the implantable medical device wall. However, tissue ingrowth may be useful for inhibiting migration of the device with respect to the implant site.

In accordance with various principles of the present disclosure, anti-migration features which are uncoated to promote tissue ingrowth therein/therearound extend from the elongate body of the implantable medical device. In some embodiments, the anti-migration features extend from at least one (and optionally both) of the retention members of the implantable medical device. In some embodiments, the anti-migration features extend transverse to the retention member, such as generally perpendicular to the retention member, to extend into tissue against which the retention member is seated. The anti-migration features may be in the form of closed or at least partially closed shapes such as U-shapes or loops. The anti-migration features may be formed with at least a retention portion extending transverse to the direction of forces against the implantable medical device causing migration thereof and/or transverse to the longitudinal axis of the implantable medical device (e.g., along which the lumen defined by the saddle region extends). In some embodiments, the anti-migration features extend from the retention members with the retention portions thereof extending generally along (e.g., in a plane generally parallel to) the plane of the retention members. As such, ingrowth of tissue along such retention portions of the anti-migration features retains the implantable medical device in place with respect to the anatomical structure from which the tissue extends and grows around the anti-migration features. It will be appreciated that even if portions of the implantable medical device (e.g., a retention member) from which the anti-migration features extend are not coated, the shape or configuration of the anti-migration features, as extending away from the outer surface of the implantable medical device and/or providing a retention portion transverse to the general direction in which migration may be expected, provide enhanced tissue ingrowth with respect to the implantable medical device.

In some embodiments, the anti-migration features are positioned at the outermost edges of the retention members. In such position, the anti-migration features leverage the maximum surface area of the retention members seated against tissue to exert retaining forces on the apposed tissue to resist migration of the stent. In some embodiments, the anti-migration features are configured to protrude or penetrate into tissue to increase their retaining hold or grip on the tissue, such as by embedding into the tissue. Tissue ingrowth in this region promotes additional anchoring of the implantable medical device with respect to the deployment site tissue. Anti-migration features formed in accordance with various principles of the present disclosure provide sufficient holding force in a gastrojejunostomy to withstand such forces as caused by peristalsis or turbulence caused by the digestion of a food bolus. As a result, an implantable medical device formed in accordance with various principles of the present disclosure has longer in-dwell duration than prior implantable medical devices.

In some aspects, an implantable medical device and its anti-migration features formed in accordance with various principles of the present disclosure may be sized, shaped, configured, dimensioned, positioned, and/or oriented to promote formation of a natural anastomosis between apposed anatomical structures by promoting tissue growth between the apposed tissue walls. More particularly, the overall dimensions of the implantable medical device (e.g., the length of the saddle region, and/or distance between the retention members) and/or the configuration of the retention members or tissue-facing surfaces thereof (such as being increasingly inclined towards the saddle region as the retention members extend radially away from the saddle region) may be selected to exert pressure to the apposed tissue walls to hold the tissue walls in apposition in a manner to further promote formation of an anastomosis therebetween.

Various embodiments of anti-migration features of implantable devices, systems, and methods in accordance with various principles of the present disclosure will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

In accordance with various principles of the present disclosure, an implantable medical device 100 configured to extend through a body passage and/or between first and second anatomical structures (e.g., tissue walls) is illustrated in FIG. 1. In the illustrated example of an embodiment, the implantable medical device 100 is shown in a gastrointestinal system, extending across an opening formed through a stomach S and a jejunum J of a patient. The implantable medical device 100 is configured to hold the walls of the stomach S and the jejunum J in close apposition to allow growth of tissue along the apposed tissues and openings therein to establish a long term and/or permanent flow or access passage therebetween. For the sake of convenience and without intent to limit, reference is made herein to an “anastomosis” and a “flow passage”, although application of principles of the present disclosure need not be so limited. In the illustrated example of an embodiment, an additional implantable medical device 1000 may be deployed across the pylorus P, such as to occlude the pylorus P and to redirect flow of materials from the stomach S through the implantable medical device 100 and into the jejunum J. It will be appreciated that principles of the present disclosure may be applied to such implantable medical device 1000 as well. As noted above, the present disclosure need not be limited to such anatomical structures, or even to the illustrated gastrointestinal environment, or even to such use. For instance, the implantable medical device 100 may be a drainage device, a support device (e.g., supporting walls of a body lumen or passage), an occlusion device (e.g., a pyloric occlusion device), etc., without intent to limit.

The example of an embodiment of an implantable medical device 100 illustrated in FIG. 1 includes an elongate body 110 having a proximal end 111 and a distal end 113. The elongate body 110 of the implantable medical device 100 may generally have a tubular configuration with a lumen 115 extending therethrough, such as between the proximal end 111 and the distal end 113 thereof. The elongate body 110 may extend the full length of the implantable medical device 100 (e.g., the proximal end 111 and the distal end 113 of the elongate body 110 may be substantially coextensive with the proximal end 101 and the distal end 103, respectively, of the implantable medical device 100), or may extend only a portion of the full extent or length of the implantable medical device 100, the present disclosure not being limited in this regard.

The implantable medical device 100 typically is shiftable between a delivery configuration and a deployed configuration. In the delivery configuration, size, shape, configuration, and/or dimensions of the implantable medical device 100 facilitate transluminal delivery thereof (e.g., through natural body passages) to an anatomical site. In the deployed configuration, the implantable medical device 100 may be sized, shaped, configured, and/or dimensioned to achieve various structures or forms for various purposes, such as to facilitate positioning of the implantable medical device 100 with respect to a treatment site, anchoring with respect thereto, formation of a passage through the anatomical site, supporting of tissue and/or tissue walls, etc. For instance, when in the delivery configuration, the elongate body 110 may have an elongated length (along a longitudinal axis LA thereof) and/or a reduced-diameter (generally transverse to the longitudinal axis LA) relative to the deployed configuration. In some embodiments, the elongate body 110 may be considered to be in a constrained, unexpanded, constricted, restrained, collapsed, etc., configuration when in the delivery configuration (not shown, but which may readily be appreciated by those of ordinary skill in the art). In the deployed configuration, the elongate body 110 may have a generally foreshortened and/or radially expanded configuration, such as relative to the delivery configuration. In some embodiments, the elongate body 110 may be considered to be in an unconstrained, expanded, unconstricted, unrestrained, neutral, etc., configuration when in the deployed configuration.

In accordance with various principles of the present disclosure, the implantable medical device 100 includes a proximal retention member 120 along a proximal end 101 of the implantable medical device 100 and a distal retention member 130 along a distal end 103 of the implantable medical device 100. It will be appreciated that although one retention member is formed along each end of the implantable medical device 100, more than one retention member may be provided on either or both ends of the implantable medical device 100. In some embodiments, the proximal end 111 of the elongate body 110 radially expands to form the proximal retention member 120, and the distal end 113 of the elongate body 110 radially expands to form the distal retention member 130, defining a saddle region 140 extending therebetween. In the deployed configuration of the implantable medical device 100, the saddle region 140 typically has a diameter greater than the diameter of the elongate body 110 in the delivery configuration. The retention members 120, 130 generally have respective diameters larger than the diameter of the saddle region 140. The diameters of the retention members 120, 130 may be the same as or different from each other, such as depending on the intended use of the implantable medical device 100 as may be appreciated by those of ordinary skill in the art. The retention members 120, 130 may be sized, shaped, configured, and/or dimensioned to anchor the implantable medical device 100 with respect to tissue walls (e.g., extending radially outwardly from a body passage through which the saddle region 140 of the implantable medical device 100 extends) to resist migration of the implantable medical device 100 with respect to the deployment site. In some embodiments, at least a portion of one or both of the retention members 120, 130 is angled towards the saddle region 140 or otherwise includes a portion or surface which protrudes towards the saddle region 140 to exert pressure against the tissue wall against which the retention member 120, 130 is positioned. The retention members 120, 130 may be formed as single-wall structures or double-wall structures. For instance, if the retention members 120, 130 are formed by expansion of proximal and/or distal portions of the wall of the implantable medical device 100/elongate body 110, such expanded wall(s) may extend radially outwardly and then return radially inwardly to form a double-wall retention member 120, 130. It will be appreciated that the retention members 120, 130 need not be limited to being a part of the ends of the elongate body 110 and may additionally or alternatively be considered extensions of the elongate body 110 at ends of the implantable medical device 100, the present disclosure not being limited in this regard.

In some embodiments (such as the example of an embodiment illustrated in FIG. 1), at least a portion of the implantable medical device 100 may advantageously be coated with a material which maintains flow of materials (such as fluids) through the lumen 115 defined through the elongate body 110 without flowing transversely through at least portions of the walls of the elongate body 110. For instance, in embodiments in which the implantable medical device 100 forms a flow passage, it may be desirable to restrict flow of materials to through the lumen 115 from the proximal end 101 to the distal end 103 of the implantable medical device 100 without leakage through/out of the lumen 115 (e.g., through the walls of the implantable medical device 100). In some embodiments, the coating is at least over portions of or the entire saddle region 140 of the implantable medical device 100 to maintain flow of materials through the saddle region 140 without flowing through the wall thereof. In some embodiments, the coating is also over additional portions of the implantable medical device 100, such as over at least portions of or all of one or both of the retention members 120, 130. It will be appreciated that the retention members 120, 130 may include respective lumens 125, 135 defined therethrough as well (such as extensions of the lumen 115 defined through the elongate body 110), and the retention members 120, 130 may also be coated with a material which maintains flow of materials (such as fluids) through the lumens 125, 135 thereof (and not through the walls of the retention members 120, 130) to flow through the lumen 115 through the elongate body 110. The coating may also provide a degree of structural stability or rigidity to the implantable medical device 100.

A coating may be provided on the implantable medical device 100 (the entire implantable medical device 100 or portions thereof) in any of a variety of manners, such as painting, dipping, spraying, sandwiching, heat shrinking, electro spinning, etc. The coating may be provided on only the outer surface or only the inner surface or both the outer surface and the inner surface of at least portions of the implantable medical device 100 wall. The coating may be any known or heretofore known biocompatible material which may prevent flow of fluids therethrough, including, without limitation, silicone, styrene isoprene butadiene (SIBS), polytetrafluoroethylene (PTFE), expanded polytetrafluoroethylene (ePTFE), ethylene tetrafluoroethylene (ETFE), fluorinated ethylene propylene (FEP), urethane, polyurethane, polyvinylidene chloride (PVC), polyether block amides (PEBA), polyimide, polyethylene, polyethylene terephthalate (PET), polysulfone, nylon, polytrimethylene terephthalate, polyvinylidene difluoride (PVDF), polyester, polyether-ester, polypropylene, polyolefin, polystyrene, polynapthalene, polyethylene napthalate (PEN), polyetherether ketone (PEEK), polyetherimide, polyphenylene sulfide (PPS), polyphenylene oxide (PPO), perfluoro(propyl vinyl ether) (PFA), polyparaphenylene teraphthalamide, polybutylene terephthalate (PBT), polyoxymethylene (POM), polyether block ester, poly(styrene-butadiene-styrene) (SBS), styrene-ethylene-butylene-styrene (SEBS), poly(styrene-b-isobutylene-b-styrene), ethylene vinyl alcohol, ethylene vinyl acetate copolymers (EVA), polycarbonates, ionomers, thermoplastic elastomers (TPE), epoxy, etc., including copolymers and/or combinations thereof.

The expandable implantable medical device may be formed in a variety of manners, such as to form a scaffold or stent structure. In some embodiments, the implantable medical device is formed from one or more members/elements (such terms being used interchangeably herein without intent to limit) combined to form a rigid and/or semi-rigid structure. The members may be formed of one or more struts, wires, strands, filaments, etc., which are braided, interengaged, intertwined, interwoven, knitted, knotted, looped (e.g., bobbinet-style), weaved, woven, wrapped, or the like to form an expandable and contractable scaffold configuration. For the sake of convenience, and without intent to limit, reference is made to filaments which are woven to form the wall of the implantable medical device 100. The filaments forming the implantable medical device may be formed from a variety of non-limiting preferably biocompatible materials, such as, without limitation, a metal, metal alloy, polymer, metal-polymer composite, ceramics, and combinations or subcombinations thereof. For instance, the filaments forming the implantable medical device may be formed from a variety of non-limiting preferably biocompatible polymers, such as, without limitation, polypropylene, polyester, polysulfone, nylon, silicones, polyurethane, polystyrene, polyethylene (PE) (including high-density and low-density PE's), polyethylene terephthalate (PET), polyethylene naphthalate (PEN), polybutylene terephthalate (PBT), polytrimethylene terephthalate, polyether block amides (PEBA), polyetheretherketone (PEEK), polyetherimide (PEI), poly(methyl methacrylate) (PMMA), polytetrafluoroethylene (PTFE), ethylene tetrafluoroethylene (ETFE), fluorinated ethylene propylene (FEP), polyoxymethylene (POM), polyether block ester, polyvinylchloride (PVC), polyvinylidene chloride (PVDC), polyether-ester, ether or ester based copolymers (for example, butylene/poly(alkylene ether) phthalate and/or other polyester elastomers, polyamides, block polyamide/ethers, polyimide (PI), ethylene vinyl alcohol, ethylene vinyl acetate copolymers (EVA), polyphenylene sulfide (PPS), polyphenylene oxide (PPO), poly praraphenylene terephthalamide, perfluoro(propyl vinyl ether) (PFA), polyolefin, epoxy, poly(styrene-b-isobutylene-b-styrene), polycarbonates, ionomers, or the like including mixtures, combinations, subcombinations, and copolymers thereof. Additionally or alternatively, the members forming the implantable medical device may be formed from a variety of non-limiting preferably biocompatible metals, such as, without limitation, stainless steel, a nickel-titanium alloy such as Nitinol, a nickel-tungsten or tungsten alloy, a cobalt-chromium alloy, a cobalt-chromium-nickel based alloy such as Elgiloy®, a nickel-copper alloy, a nickel-cobalt alloy, a nickel-iron alloy, a nickel-chromium alloy, a nickel-molybdenum alloy, a nickel-chromium-molybdenum alloy, a nickel-cobalt-chromium-molybdenum alloy, a cobalt-chromium-molybdenum alloy, platinum enriched stainless steel, titanium, or the like, including combinations and subcombinations and other alloys thereof. Additionally or alternatively, the members forming the implantable medical device may be formed from a variety of non-limiting preferably biocompatible natural materials, such as, without limitation, cat or bovine intestine, or the like; a natural fiber, such as silk or cotton, or the like, and combinations and subcombinations thereof. It will be appreciated that the members forming the implantable medical device may be formed from a mixtures, composites, combinations, subcombinations, copolymers, or co-constructions of any of the above. Alternatively, the members forming the implantable medical device may be formed by cutting (e.g., by laser-cutting) a tubular structure (e.g., an, optionally monolithic, cylindrical tubular member) into an expandable configuration, the cuts forming members such as strut members. The implantable medical device may be a self-expanding device such as known or heretofore known to those of ordinary skill in the art. For instance, the implantable medical device may be formed of shape-memory or heat-formable material (e.g., Nitinol or Elgiloy® or shape memory polymers) so that the implantable medical device returns to a pre-shaped expanded configuration from a collapsed configuration upon advancement from a delivery sheath (any acceptable tubular elongated member such as known to those of ordinary skill in the art for delivery of medical devices) and/or withdrawal of a delivery sheath which maintains the implantable medical device in a delivery configuration therein.

In some embodiments of implantable medical devices formed in accordance with various principles of the present disclosure, such as those formed as a braided or woven element, the walls of the implantable medical device have gaps, apertures, openings, interstices, etc. therethrough. Application of a coating material to, over, on, etc. (such terms being used interchangeably herein without intent to limit) may fill in such gaps, apertures, openings, interstices, etc., in the walls of the implantable medical device to inhibit or prevent flow or leakage of materials therethrough. In some embodiments, the coating may encapsulate members forming the wall of the implantable medical device 100. For instance, the elements forming the wall of an example of an embodiment of an implantable medical device 100 formed in accordance with various principles of the present disclosure may be embedded in the coating material, such as to be fully coated by the coating material.

Although the coating of an implantable medical device formed in accordance with various principles of the present disclosure provides various benefits, it may be appreciated that such coating may be lubricious or otherwise may be slippery or otherwise resistant to maintaining a desired position of the implantable medical device 100 with respect to body tissue along which the implantable medical device 100 is deployed. In accordance with various principles of the present disclosure, one or more anti-migration features 150 extend along or from any portion or portions of the implantable medical device 100 where such location of an anti-migration feature 150 may facilitate holding (e.g., anchoring) of the implantable medical device 100 in place to resist migration from the deployment site. It will be appreciated, as described in further detail below, that the anti-migration features 150 are described herein as extending or formed or provided, on or along or about an implantable medical device 100, such descriptive terms (and other grammatical forms thereof, including reasonable alternatives of such terms) being used interchangeably herein and without intent to limit. The anti-migration features 150 may be sized, shaped, configured, dimensioned, positioned, located, etc., in a variety of manners to increase resistance of the implantable medical device 100 against migration from the implant site, which may be appreciated by those of ordinary skill in the art in view of the following. Typically, the anti-migration features 150 are provided along an outer surface of the implantable medical device 100 which faces tissue so that the anti-migration features 150 interact with the tissue to hold the implantable medical device 100 with respect to the tissue to resist migration therefrom.

In some aspects, the anti-migration features 150 are not coated or are otherwise not provided with a coating as provided on other portions of the implantable medical device 100, particularly along the saddle region 140). In accordance with various principles of the present disclosure, the uncoated anti-migration features 150 are provided along one or more portions or areas of the implantable medical device 100 facing tissue at the deployment site. The lack of coating on the anti-migration features 150 allows and may even promote tissue ingrowth around the anti-migration features 150 so that the implantable medical device 100 is held in place with respect to the deployment site.

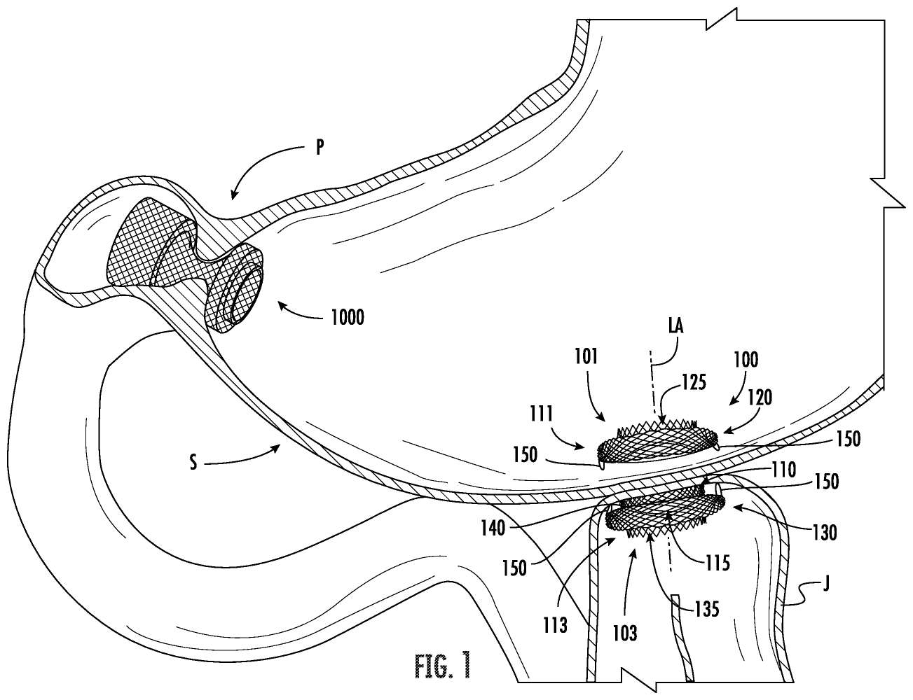

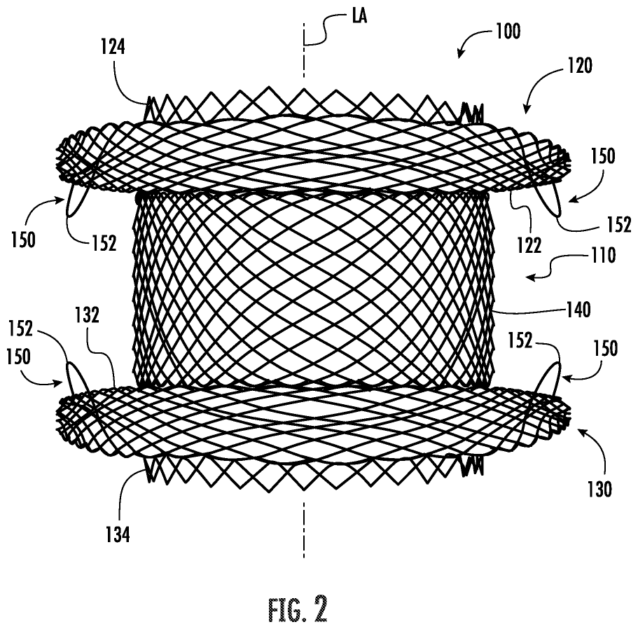

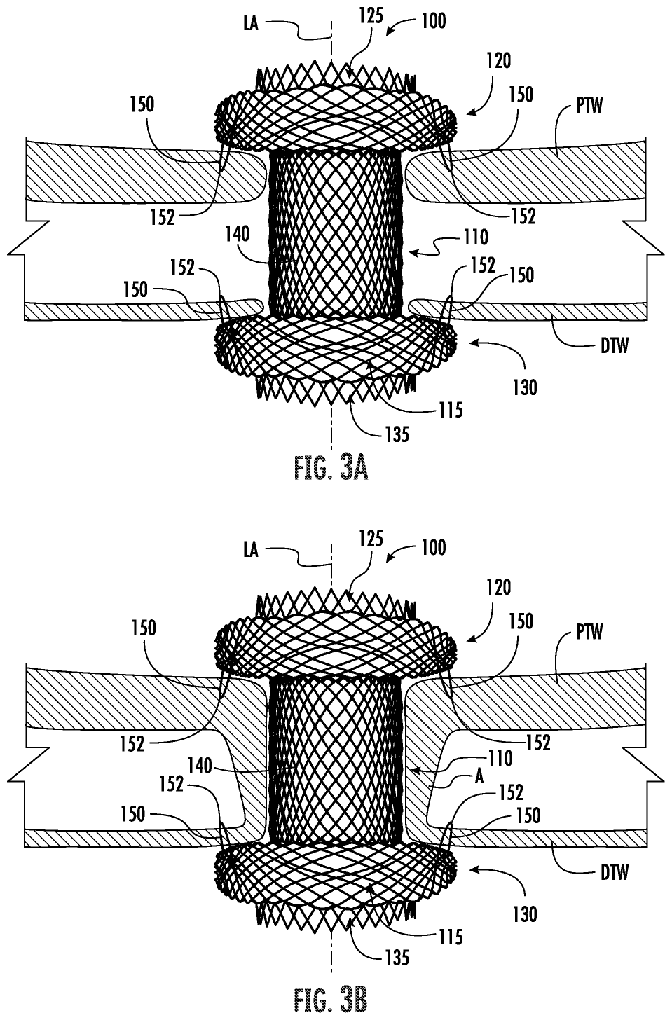

In some embodiments, at least one of the anti-migration features 150 includes a retention portion 152 positioned and oriented to withstand forces which impact the implantable medical device 100. For instance, the retention portion 152 may extend transverse to a direction of potential migration of the implantable medical device 100 and/or transverse to the primary direction in which forces may impact the implantable medical device 100. In the examples of embodiments illustrated in FIG. 1, FIG. 2, FIG. 3A, and FIG. 3B, the anti-migration features 150 are U-shaped or loops or otherwise formed as closed or semi-closed shapes so that tissue grows around and through such shape to resist migratory forces on the implantable medical device 100 (e.g., from normal bodily movements or functions, such as peristalsis, or passage of materials through the anastomosis formed by and maintained along the implantable medical device 100). In the examples of embodiments illustrated in FIG. 1, FIG. 2, FIG. 3A, and FIG. 3B, one or more of the anti-migration features 150 include at least one retention portion 152 extending transverse to a direction of potential migration of the implantable medical device 100 and/or transverse to the primary direction in which forces may impact the implantable medical device 100. For instance, the retention portion 152 may be the portion of a U-shaped anti-migration features 150 extending transverse to/coupling the legs (e.g., generally parallel portions) of the U-shaped anti-migration features 150 (the legs extending to and engaging a retention member 120, 130 of the implantable medical device 100). In the examples of an embodiment illustrated in FIG. 1, the implantable medical device 100 typically is subjected the greatest forces (within the anatomical site at which it is deployed) along the longitudinal axis LA thereof. Accordingly, as may be appreciated with reference to the example of an embodiment of an implantable medical device 100 illustrated in FIG. 2, FIG. 3A, and FIG. 3B, the retention portion 152 extends generally transverse to the longitudinal axis LA of the implantable medical device 100. As such, as tissue grows around the retention portion 152 of an anti-migration feature 150, the anti-migration feature 150 and thus the implantable medical device 100 are increasingly more securely held in place with respect to the tissue at the deployment site.

In accordance with various principles of the present disclosure, one or more, such as two or more, or three or more, or more than three anti-migration features 150 may extend from a portion of the implantable medical device 100 and/or different portions of the implantable medical device 100. The anti-migration features 150 may be spaced apart from one another along a region or section of the implantable medical device 100 (e.g., along one of the retention members 120, 130). It will be appreciated that various configurations and/or locations of anti-migration features 150 with respect to an implantable medical device 100 formed in accordance with various principles of the present disclosure are within the scope and spirit of the present disclosure, such as described in further detail below with reference to examples of embodiments. For instance, the number of anti-migration features 150 may be increased or decreased, one or more anti-migration features 150 may be in close proximity of one another (e.g., staggered near one another), the locations of the anti-migration features 150 may be varied, the shapes of the anti-migration features 150 may be varied, the relative proportions of the anti-migration features 150 and implantable medical device 100 may be varied, the dimensions of the anti-migration features 150 may be varied, etc.

In the examples of embodiments illustrated in FIG. 1, FIG. 2, FIG. 3A, and FIG. 3B, one or more anti-migration features 150 extend from at least one of the retention members 120, 130. Optionally, one or more anti-migration features 150 are provided on both the proximal retention member 120 and the distal retention member 130. Provision of at least one anti-migration feature 150 on both retention members 120, 130 may increase the anti-migratory impact of the anti-migration features 150 by resisting forces on the implantable medical device 100 which are directed distally as well as proximally. The anti-migration features 150 may be positioned along various locations along the retention members 120, 130, along the periphery of the retention members 120, 130 or anywhere along the surface of the retention members 120, 130, spaced apart or staggered near one another.

In some embodiments, such as illustrated in FIG. 1, FIG. 2, FIG. 3A, and FIG. 3B, the one or more anti-migration features 150 extend from a radially-extending wall 122, 132 of at least one of the retention members 120, 130. As referenced herein, the radially-extending walls 122, 132 are the walls forming the retention members 120, 130 which are generally transverse to the longitudinal axis LA of the implantable medical device 100. The radially-extending walls 122, 132 of the retention members 120, 130 are generally also transverse to the saddle region 140 which generally extends along the longitudinal axis LA of the implantable medical device 100. Such configuration is advantageous when the implantable medical device 100 is deployed at anatomical sites where forces within the body impact the implantable medical device 100 along the longitudinal axis LA thereof. For instance, if the implantable medical device 100 defines a flow passage therethrough (e.g., through a lumen 115 defined through the implantable medical device 100), forces which may cause migration of the implantable medical device 100 are typically greatest along the longitudinal axis LA thereof. Thus, the radially-outwardly extending walls of the retention members 120, 130, as transverse to the longitudinal axis LA of the implantable medical device 100, provide increased surface area to resist axial forces on the implantable medical device 100. The radially-outwardly extending walls of the retention members 120, 130 may anchor implantable medical device 100 against the tissue walls extending (e.g., radially outwardly) from the passage through which the implantable medical device 100 is deployed and/or the tissue walls against which the retention members 120, 130 are deployed. In some embodiments, the anti-migration features 150 are positioned along the radially outermost edge of the retention members 120, 130. Such position of the anti-migration features 150 may be beneficial in contributing to retaining the greatest surface area of the radially-extending walls 122, 132 of the retention members 120, 130 being pressed against tissue. However, other locations are within the scope and spirit of the present disclosure.

The anti-migration features 150 may extend substantially against or along or parallel to or generally flat with respect to the wall of the implantable medical device 100 when the implantable medical device 100 is in a delivery configuration to allow a compact configuration thereof. However, in a deployed configuration of the implantable medical device 100, the anti-migration features 150 may extend at various angles (i.e., angles greater than 0 degrees and less than 180 degrees) with respect to the wall of the implantable medical device 100 from which the anti-migration features 150 extend. For instance, the anti-migration features 150 may extend from a wall of the implantable medical device 100 in a direction transverse to such wall. For instance, in the example of an embodiment illustrated in FIG. 2, the anti-migration features 150 extend from and generally transverse to the respective radially-extending walls 122, 132 of the retention members 120, 130 along the side of the retention members 120, 130 facing towards the saddle region 140. Thus, the anti-migration features 150 extend from the retention members 120, 130 towards the saddle region 140. The anti-migration features 150 may extend towards the saddle region 140 at an approximately 45° to about 135° angle with respect to a wall 122, 132 of a retention member 120, 130. In other words, the anti-migration features 150 may be perpendicular or up to about 45° from perpendicular with respect to a wall 122, 132 of a retention member 120, 130. If inclined with respect to (i.e., not perpendicular to) a wall 122, 132 of a retention member 120, 130, the anti-migration features 150 may be inclined toward or away from the saddle region 140. The angle at which an anti-migration feature 150 extends with respect to a retention member 120, 130 could vary in either direction (inwardly or outwardly), such as, without limitation, based on the amount of pressure desired to be exerted on the gastric wall, and/or general characteristics of the deployment site, and/or a desire to promote tissue ingrowth away from the saddle region 140 (typically outwardly) or closer to the saddle region 140 (typically inwardly).

In some embodiments, the anti-migration features 150 may be configured to extend into the walls of the anatomical tissue along which the implantable medical device 100 is deployed. It will be appreciated that terms such as penetrate, anchor, bite, embed, etc., and other grammatical forms thereof, may be used interchangeably herein without intent to limit. As illustrated in FIG. 3A, an example of an embodiment of an implantable medical device 100 is extended between a proximal tissue wall PTW and a distal tissue wall DTW. Specifically, the elongate body 110 of the implantable medical device 100 extends from a proximal side of the proximal tissue wall PTW to the distal side of the distal tissue wall DTW with the proximal retention member 120 positioned against the proximal side of the proximal tissue wall PTW and the distal retention member 130 positioned against the distal side of the distal tissue wall DTW. In the illustrated example of an embodiment, anti-migration features 150 extend transversely from the retention members 120, 130 and towards the saddle region 140 of the implantable medical device 100. The length of the elongate body 110, particularly of the saddle region 140 thereof, may be selected so that the retention members 120, 130 exert pressure against the tissue walls PTW and DTW to hold the tissue walls PTW and DTW in apposition. Such pressure may cause the anti-migration features 150 to embed into the tissue walls PTW and DTW to further enhance the anti-migratory nature of the anti-migration features 150. As the tissue walls PTW and DTW are held in apposition, tissue may grow along the saddle region 140 of the implantable medical device 100 to form an anatomical/tissue (as opposed to an artificial, such as formed by the implantable medical device 100) anastomosis, such as illustrated in FIG. 3B. In some instances, tissue growth along the saddle region 140 may even be encouraged, particularly if pressure exerted by the retention members 120, 130 (optionally enhanced by the anti-migration features 150) is exerted against the apposed tissues, such as causing the apposed tissues to fuse together.

It will be appreciated that the anti-migration features 150, may be formed in any of a variety of manners in accordance with various principles of the present disclosure. The anti-migration features 150 may be formed as wires or filaments or threads or tethers or ropes or bands or other elements providing sufficient resistance to forces impacting the implantable medical device 100 and the anti-migration features 150. The anti-migration features 150 may be formed separately from the implantable medical device 100 and coupled (directly or indirectly) thereto, such as by welding, soldering, interweaving, adhering (e.g., gluing), mechanically deforming (e.g., knotting, looping, crimping, interference or friction fitting, etc.), or other manners known to those of ordinary skill in the art. Additionally or alternatively, the anti-migration features 150 may be integral extensions of the walls of the implantable medical device 100. For instance, in some embodiments, one or more of the elements forming the implantable medical device 100 (e.g., woven or interwoven filaments) may be extended or pulled away from the remainder of the implantable medical device 100 to form an anti-migration feature 150. More particularly, a lip 124, 134 may extend axially from the retention members 120, 130 in a direction away from the saddle region 140 with sufficient additional material to allow a filament (or other member) of the implantable medical device 100 to be pulled to form anti-migration features 150 therefrom. The manner in which the anti-migration features 150 are formed is selected to enhance the anti-migratory forces to be achieved by the anti-migration features 150. For instance, the manner in which the anti-migration features 150 are formed may be selected to facilitate formation of a retention portion 152 such as described above.

In view of the above descriptions, it will be appreciated that the devices, systems, and methods disclosed herein can be used to form one or more anastomoses, and can be used with basic endoscopic tools, catheters, laparoscopes, general surgery tools, etc. For example, a catheter-based stent delivery device can be used with an endoscope to form one anastomosis, for example between two portions of the intestines. An endoscopic-based device could be used to form an anastomosis between the fundal pouch and a portion of the intestines, such as the jejunum. A combination of a laparoscopic-based device and a catheter-device as described herein could also be used to form a single anastomosis. When deploying a stent or other tissue anchor between adjacent body lumens, organs, or other structures, it is typically necessary to penetrate both a first tissue wall (e.g., a wall of an organ or a first body lumen), through which access is established, and a second tissue wall (e.g., of a wall of an organ or a second body lumen) which is the target for the procedure. For instance, an instrument may be introduced to an anatomical site at which the anastomosis is to be performed to form (e.g., cut) a passage between adjacent tissues. Tissue at the deployment site may be pretreated in a variety of manners, such as by abrasion (e.g., with the use of a hook knife, hot biopsy forceps, and hot snares), ablation, pharmaceutical treatments, argon plasma coagulation (APC), etc., to promote, accelerate, and/or increase cellular growth, such as a result of a healing response. The induced tissue growth may facilitate the above-described tissue ingrowth into the anti-migration features of the implantable medical device to be positioned at the treated site. A delivery device (e.g., tubular elongate member) may then be navigated to the anatomical site with the implantable medical device therein. The distal end may be extended from the delivery device and/or the delivery device may be retracted to deploy a distal end of the device. In some embodiments, the distal end expands to form a retention member anchoring the implantable medical device with respect to the distal tissue wall. The delivery device may then be further withdrawn to expose the portions of the implantable medical device proximal to the distal end of the implantable medical device. The proximal end of the implantable medical device may expand to form a proximal retention member anchoring the implantable medical device with respect to the proximal tissue wall.

It will be appreciated that the present disclosure is not to be limited to a particular form or configuration of an implantable medical device, or system or method used therewith, principles of the present disclosure being applicable to various configurations of implantable medical devices, systems, and methods such as known to those of ordinary skill in the art. It will be appreciated that various aspects of the above disclosure may be applied to other implantable medical devices, systems, and/or methods, such as devices positioned in other locations within the body, whether or not a flow passage exists or is created at such location.

Although embodiments of the present disclosure may be described with specific reference to medical devices and systems and procedures for forming an anastomosis, it will be appreciated that principles of the present disclosure may be applied to devices such as the implantable medical device 1000 illustrated in FIG. 1 for forming a gastric outlet obstruction (e.g., occluding a pylorus P). Moreover, although embodiments of the present disclosure are described with specific reference to medical devices and systems and procedures for treating the gastrointestinal system, it should be appreciated that such medical devices and methods may be used with implantable medical devices used in the abdominal cavity, digestive system, biliary system, urinary tract, reproductive tract, respiratory system, cardiovascular system, circulatory system, etc.

Various further benefits of the various aspects, features, components, and structures of anti-migration features such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An implantable medical device comprising:
an elongate body having a proximal end and a distal end and defining a lumen extending therethrough;
a proximal retention member along the proximal end of said elongate body;
a distal retention member along the distal end of said elongate body;
a saddle region defined between said proximal retention member and said distal retention member; and
at least one anti-migration feature extending outwardly from an outer surface of said elongate body;
wherein:
at least said saddle region has a wall with a coating formed of a material preventing flow of fluid therethrough; and
said at least one anti-migration feature is uncoated to promote tissue ingrowth therearound.

2. The implantable medical device of claim 1, wherein said at least one anti-migration feature is in the form of a semi-closed or closed shape with respect to said elongate body.

3. The implantable medical device of claim 2, wherein said at least one anti-migration feature includes at least one retention portion extending transverse to an axis along which migratory forces impact said implantable medical device.

4. The implantable medical device of claim 3, wherein the lumen of said elongate body extends through said saddle region and said retention portion extends transverse to the longitudinal axis of said saddle region.

5. The implantable medical device of claim 4, wherein said at least one anti-migration feature extends from and transverse to at least one of said proximal retention member or said distal retention member in a direction toward said saddle region.

6. The implantable medical device of claim 1, wherein:
said proximal retention member and said distal retention member extend radially outwardly from said saddle region; and
said at least one anti-migration feature extends from and transverse to at least one of said proximal retention member or said distal retention member in a direction toward said saddle region.

7. The implantable medical device of claim 6, wherein said saddle region is configured to extend between a proximal tissue wall and a distal tissue wall, said proximal retention member is configured to anchor said implantable medical device with respect to the proximal tissue wall, the distal retention member is configured to anchor said implantable medical device with respect to the distal tissue wall, and said at least one anti-migration feature is configured to embed into one of the proximal tissue wall or the distal tissue wall.

8. The implantable medical device of claim 7, wherein said at least one anti-migration feature includes at least one anti-migration feature extending from said proximal retention member toward said saddle region and at least one anti-migration feature extending from said distal retention member toward said saddle region.

9. The implantable medical device of claim 1, wherein:
said implantable medical device is shiftable between an elongated delivery configuration and a foreshortened deployed configuration;
said proximal retention member and said distal retention member are defined upon said implantable medical device shifting to the deployed configuration and a portion of said elongate body extending radially outward; and
in the foreshortened configuration, the length of said saddle region and the configuration of said proximal retention member and said distal retention member are selected to draw together tissues across which said elongate body is extended, with said at least one anti-migration feature anchoring into the tissues.

10. The implantable medical device of claim 1, wherein at least one of said proximal retention member or said distal retention member is formed from woven filaments, and said at least one anti-migration feature is formed from an extension of one of said woven filaments.

11. The implantable medical device of claim 1, wherein said at least one anti-migration feature is separately formed from and coupled to at least one of said proximal retention member or said distal retention member.

12. An implantable medical device comprising:
an elongate body having a proximal end and a distal end and defining a lumen extending therethrough; and
at least one anti-migration feature extending outwardly from an outer surface of said elongate body;
wherein:
said elongate body is formed from a plurality of filaments forming a wall of said elongate body with interstices therethrough;
at least a portion of said wall of said elongate body is coated to prevent flow of fluid therethrough and to resist tissue ingrowth therein; and
said at least one anti-migration feature is uncoated to promote tissue ingrowth therearound.

13. The implantable medical device of claim 12, wherein said at least one anti-migration feature is in the form of a semi-closed or closed shape with respect to said elongate body

14. The implantable medical device of claim 13, wherein said at least one anti-migration feature includes at least one retention portion extending transverse to an axis along which migratory forces impact said implantable medical device.

15. The implantable medical device of claim 12, wherein:
said implantable medical device further comprises a proximal retention member extending radially outwardly along the proximal end of said elongate body, a distal retention member extending radially outwardly along the distal end of said elongate body, and a saddle region defined between said proximal retention member and said distal retention member; and
said at least one anti-migration feature extends from at least one of said retention members towards said saddle region.

16. A method for creating an anastomosis, said method comprising:
extending an implantable medical device across a proximal tissue wall and a distal tissue wall;
allowing a proximal end of the implantable medical device to expand radially outwardly with respect to a saddle region extending through the tissue walls to form a proximal retention member anchored against a proximal side of the proximal tissue wall; and
allowing a distal end of the implantable medical device to expand radially outwardly with respect to the saddle region to form a distal retention member anchored against distal side of the distal tissue wall;
wherein:
the saddle region has a tubular wall defining a lumen therethrough, the wall being coated with a material preventing passage of fluid therethrough; and
said method further comprises positioning the implantable medical device so that at least one uncoated anti-migration feature extends towards at least one of the proximal tissue wall or the distal tissue wall and tissue ingrowth with respect to the at least one anti-migration feature is promoted.

17. The method of claim 16, further comprising causing the at least one anti-migration feature to embed into the at least one of the proximal tissue wall or the distal tissue wall.

18. The method of claim 17, further comprising deploying the implantable medical device such that the proximal retention member and the distal retention member draw the proximal tissue wall and the distal tissue wall together in apposition to facilitate formation of an anastomosis therebetween.

19. The method of claim 16, further comprising positioning the implantable medical device so that at least one uncoated anti-migration feature extends from at least one of the proximal retention member or the distal retention member and towards a respective proximal or distal tissue wall to promote tissue ingrowth with respect to the at least one uncoated anti-migration feature.

20. The method of claim 19, further comprising positioning the implantable medical device so that at least one proximal uncoated anti-migration feature extends transverse to and from the proximal retention member to the proximal tissue wall to promote tissue ingrowth with respect to the at least one proximal uncoated anti-migration feature, and so that at least one distal uncoated anti-migration feature extends transverse to and from the distal retention member and towards the distal tissue wall to promote tissue ingrowth with respect to the at least one distal uncoated anti-migration feature.
